(19) United States
(12) Patent Application Publication
(10) Pub. No.: US 2022/0339369 A1
(43) Pub. Date: Oct. 27, 2022

(54) NEEDLE SYSTEM FOR DRUG INJECTION

(71) Applicant: Novo Nordisk A/S, Bagsvaerd (DK)

(72) Inventors: Henrik Bengtsson, Taastrup (DK); Ronan James Carroll, Daleen (IE); Vera Pinto Glenting, Copenhagen (DK)

(21) Appl. No.: 17/761,330

(22) PCT Filed: Sep. 22, 2020
(86) PCT No.: PCT/EP2020/076432

§ 371 Date: Mar. 17, 2022

(30) Foreign Application Priority Data
Sep. 24, 2019 (EP) 19199276.7
Nov. 22, 2019 (EP) 19210845.4
Dec. 20, 2019 (EP) 19218778.9

(51) Int. Cl. A61M 5/32

(52) U.S. Cl. CPC A61M 5/3271; A61M 5/3293; A61M 5/3286; A61M 2205/0205; A61M 2005/247

(57) ABSTRACT

the present invention provides a needle system comprising a needle assembly (1, 100, 200, 300) and a drug cartridge (30, 130, 230, 330), the needle assembly (1, 100, 200, 300) comprising a needle hub member (4.1, 104.1, 204.1, 304.1) in which an injection needle (15, 115, 215, 315) is fixedly mounted such that a front needle portion (15f, 115f, 215f, 315f) extends in a distal direction therefrom and a rear needle portion (15r, 115r, 215r, 315r) extends in a proximal direction therefrom, and a skirt member (4.2, 104.2, 204.2, 304.2) extending proximally from the needle hub member (4.1, 104.1, 204.1, 304.1) and defining a skirt cavity (4.9, 104.9, 204.9, 304.9) accommodating the rear needle portion (15r, 115r, 215r, 315r), the skirt cavity (4.9, 104.9, 204.9, 304.9) having a skirt cavity configuration, and the drug cartridge (30, 130, 230, 330) comprising a generally cylindrical body (31, 131, 231, 331) being sealed proximally by a slidable piston (239, 339) and having an outlet end portion defined by a neck (32, 132, 232, 332) and a head (33, 133, 233, 333), the head (33, 133, 233, 333) having a head configuration, wherein the skirt cavity configuration corresponds to the head configuration, providing for a tight fitting of the skirt member (4.2, 104.2, 204.2, 304.2) on the head (33, 133, 233, 333) which renders the rear needle portion (15r, 115r, 215r, 315r) a permanent resident of the outlet end portion.

### FIELD OF THE INVENTION

The present invention relates to needle systems for use in drug injection procedures.

### BACKGROUND OF THE INVENTION

Injection systems for self-administering of drugs comprising a pen injection device and an attachable needle unit have become increasingly popular due to generally simple and convenient handling patterns.

Predominantly, two types of pen injection devices are available, durable injection devices being capable of delivering one or more doses of drug from a prefilled drug cartridge which can be loaded into the device before use and replaced after exhaustion, and disposable injection devices being capable of delivering one or more doses of drug from a prefilled and non-exchangeable drug cartridge. Each of these types of pen injection devices are, or may in principle be, realised in various sub-types, such as e.g. single shot devices adapted to deliver only one dose of a predetermined, or selected, size from a drug cartridge, multi-shot devices capable of delivering a plurality of doses from a drug cartridge, manual devices, where the user provides the force needed for injection, automatic devices having a built-in energy source releasable to occasion the injection, fixed dose devices adapted to deliver doses of identical size, variable dose devices offering delivery of a plurality of doses of drug, each settable by the user from a range of possible dose sizes, etc.

As the labels suggest a durable injection device is intended for use over a considerable period of time during which multiple drug cartridges are exhausted and replaced, whereas a disposable injection device is intended for use until its dedicated drug cartridge is exhausted, after which the entire injection device is discarded.

Regardless of the type of pen injection device users of such injection systems are recommended to discard the needle unit after a single injection to minimise the risk of contamination. Hence, in the course of its lifetime a multi-use injection device is by default to be used with multiple needle units.

Needle units are typically wrapped and sealed individually to ensure sterility prior to use. In connection with a dose administration action the user must therefore unwrap the needle unit, mount it on the injection device to thereby establish fluid connection to the drug cartridge, perform the injection, dismount it from the injection device, re-wrap or otherwise encapsulate it to prevent needle stick injuries, and finally dispose of it, preferably in a dedicated sharps container.

The readying and subsequent removal of the needle unit is both the most complicated and the most time-consuming part of the injection procedure. With a traditional thread interface the mounting and dismounting of the needle unit even require users to perform a repetitive rotary hand movement which some find motorically difficult. In general, especially for young and elderly users the handling of the small items and foils can present a challenge and make the task of injection cumbersome.

WO 2015/117854 (Novo Nordisk A/S) discloses a combined cartridge and needle assembly for use in a re-usable injection device. The cartridge containing a liquid drug and the needle assembly are permanently attached to each other to form a single drug unit which is insertable in the injection device. The needle assembly comprises a housing, a telescopic needle shield and an axially movable needle hub which is moved axially to an injection position by a resilient element, once the combined cartridge and needle assembly is inserted in the injection device, upon rotation of the housing. The movement of the needle hub establishes fluid connection to the cartridge, creates a reservoir within the needle shield between two separable septa which are axially drawn apart, and generates a vacuum inside the reservoir which resultantly initiates a transfer thereto of a volume of the liquid drug from the cartridge. When the reservoir is thus filled, the injection device is ready to perform a first of a plurality of injections possible without change of the needle assembly, due to preservatives in the liquid drug cleaning the needle tip in the reservoir after each injection action, and when the cartridge is eventually empty, the drug unit can be replaced.

While this solution does ease the readying of the injection device and does reduce the number of user actions normally performed in connection with the needle handling, it still requires both an insertion of the drug unit into the injection device as well as a preparative rotation of the housing to cause the movement of the needle hub which leads to the establishment of fluid connection to the cartridge and the subsequent filling of the reservoir. Even though the filling of the reservoir is automatic and therefore does not require further actions from the user it prolongs the time necessary to ready an injection device with a new drug unit for injection. Furthermore, the solution entails a comparably complex needle assembly construction involving several movable parts, increasing the risk of malfunction.

### SUMMARY OF THE INVENTION

It is an object of the invention to eliminate or reduce at least one drawback of the prior art, or to provide a useful alternative to prior art solutions.

In particular, it is an object of the invention to provide a solution which reduces the number of handling steps associated with the use of a needle-based injection system.

It is a further object of the invention to provide a solution which enables a person to quickly and easily ready a needle-based injection system for use

It is a further object of the invention to provide such a solution which is simple, compact and cost-effective.

It is a further object of the invention to provide such a solution which is safe and reliable.

In the disclosure of the present invention, aspects and embodiments will be described which will address one or more of the above objects and/or which will address objects apparent from the following text.

In a first aspect the invention provides a needle system according to claim 1.

Accordingly, a needle system is provided comprising a needle assembly and a drug cartridge. The needle assembly comprises a needle hub member in which an injection needle, extending along a reference axis, is fixedly mounted, e.g. by gluing. A front needle portion of the injection needle extends in a distal direction from the needle hub member, and a rear needle portion of the injection needle extends in a proximal direction from the needle hub member. The needle assembly further comprises a skirt member which extends proximally from the needle hub member and defines a skirt cavity accommodating the rear needle portion. The skirt cavity has a skirt cavity configuration, i.e. a certain size and shape.

The drug cartridge is a conventional type drug cartridge and comprises a generally cylindrical cartridge body and a slidable piston which define a chamber for holding a liquid substance. The cartridge body has an outlet end portion defined by a necked down section, or neck, and a head. The head has a head configuration which corresponds to the skirt cavity configuration.

This provides for a firm and stable attachment of the needle assembly to the drug cartridge by a tight fitting of the skirt member on the head, ensuring that the rear needle portion resides permanently in the outlet end portion in the sense that the rear needle portion is fluidly connected with the chamber at all times. Thereby, an assembled needle system can be inserted into an injection device and a force can be applied to the piston to expel a dose of the liquid substance immediately without any further preparatory user actions.

The tight fitting of the skirt member on the head additionally provides for a compact construction of the needle system and gives a solid sense of unitarity because there is no intermediate space to allow for wriggling. Thus, because the skirt cavity configuration corresponds to the head configuration, in the assembled needle system the skirt member and the head are firmly engaged such that no relative axial or tilting motion between the needle assembly and the drug cartridge is possible.

The needle assembly and the drug cartridge may be pre-assembled, e.g. by the manufacturer, and the needle system may thus be offered to the user in a pre-assembled state, e.g. in a sterile packaging which is easily removable to enable quick insertion into the injection device. The needle system may e.g. be designed to be front-loaded into a dedicated cartridge holder, or to be transversally clicked into a dedicated cartridge holder.

The skirt member and the needle hub member may be formed in one piece, whereby a particularly simple and cost-effective construction is obtained.

In some embodiments of the invention the head comprises a self-sealing septum sealing the outlet end portion, and an end cap for immobilising the self-sealing septum with respect to the generally cylindrical body. This allows for manufacturing of the needle assembly and the drug cartridge as separate entities.

In other embodiments of the invention the skirt member further comprises a pre-arranged self-sealing septum, and the rear needle portion transpierces a central portion of the self-sealing septum. The skirt cavity configuration thus corresponds to the head configuration of an open drug cartridge, and the outlet end portion will become sealed upon fitting of the skirt member on the head. Thereby, an end cap for immobilising the self-sealing septum is omitted, reducing the number of components needed for the needle system. It is noted that to provide an enhanced sealing effect the self-sealing septum may be slightly compressible, and in cases where the skirt cavity configuration corresponds to the head configuration of an open drug cartridge, this should be interpreted to include the skirt cavity configuration being slightly smaller than the head configuration to account for the compression of the self-sealing septum as the head enters the skirt cavity.

The skirt member may be configured to snap to the drug cartridge behind the head. In particular, the head may comprise a trailing rim and the skirt member may comprise a catch portion adapted to engage with the trailing rim. This will provide a very compact construction of the needle system.

The needle system may further comprise a removable plug member fitted tightly around a distal end of the front needle portion. Such a plug member will be able to serve a dual purpose of maintaining sterility of the section of the front needle portion that goes into the skin of the user and preventing leakage of the liquid substance from the drug cartridge through the injection needle. The removable plug member may be adapted to be slid along the distal end of the front needle portion.

While the above described needle system may be particularly suitable for use in a single shot type of injection device, where the drug cartridge is emptied and discarded after one injection action, it may be used in a multi-shot type of injection device capable of delivering a plurality of doses from the drug cartridge as well, if the distal end of the front needle portion is cleaned after each injection action. This could for example be accomplished by the removable plug member comprising a micro-bacterial growth inhibitor and the user remounting the removable plug member on the distal end of the front needle portion after each use of the injection device.

However, alternatively, the needle assembly may further comprise a, non-removable, plug member fitted tightly around a portion of the front needle portion, the plug member comprising a penetrable self-sealing front section and being displaceable, along a longitudinal axis of the injection needle, with respect to the needle hub between a forward plug position in which a distal end section of the front needle portion is housed within the plug member, proximally of the penetrable self-sealing front section, and a rearward plug position in which the distal end section is exposed. Since the plug member forms an integral part of the needle assembly the risk of the user losing it is eliminated. Consequently, the plug member is always available and positionable to prevent accidental needle stick injuries.

Also in this case the plug member may comprise a micro-bacterial growth inhibitor. In particular, the plug member may be made of a thermoplastic elastomer containing immobilised Zinc (Zi⁺⁺) or immobilised silver (Ag⁺⁺), as these ions are known to inhibit micro-bacterial growth. Since the plug member fits tightly around a portion of the front needle portion any micro-bacterial contaminants thereon will resultantly be neutralised. With the plug member in the forward plug position between use the distal end section is thereby kept in a biostatic environment between injections and the needle assembly is accordingly suitable for multiple use.

The plug member may further comprise a cylindrical plug body defining a solid plug portion with a cylindrical channel therein. In that case the cylindrical channel borders on the self-sealing front section and has a transversal channel dimension which is smaller than a transversal dimension of the injection needle.

The plug member may be axially displaceable with respect to the needle hub member by translation or by helical motion. In either case, the plug member may be biased towards the forward plug position, e.g. by a resilient element such as a spring.

Hence, the plug member may be displaced proximally from the forward plug position to the rearward plug position by the user placing the penetrable self-sealing front section on, and pressing the needle system against, the skin to insert the distal end section of the front needle portion and distally from the rearward plug position to the forward plug position by the resilient element upon removal of the injection needle from the skin.

In some embodiments of the invention the needle assembly further comprises means for displacing the plug member with respect to the needle hub member. For example, the needle assembly may further comprise a sleeve member rotatable relative to the needle hub member between a first angular position and a second angular position, and may be configured to cause a) a displacement of the plug member from the forward plug position to the rearward plug position in response to a rotation of the sleeve member from the first angular position to the second angular position and b) a displacement of the plug member from the rearward plug position to the forward plug position in response to a rotation of the sleeve member from the second angular position to the first angular position.

In a second aspect the invention provides a needle assembly for attachment to a drug cartridge comprising a generally cylindrical body being sealed proximally by a slidable piston and having an open outlet end portion defined by a neck and a head, the needle assembly comprising a needle hub member in which an injection needle is fixedly mounted such that a front needle portion extends in a distal direction therefrom and a rear needle portion extends in a proximal direction therefrom, a skirt member extending proximally from the needle hub member and defining a skirt cavity for reception of the head, and a self-sealing septum, wherein the rear needle portion extends into the skirt cavity through the self-sealing septum.

This allows for a combination of the needle assembly with the drug cartridge by insertion of the head into the skirt cavity, thereby producing a needle system for use with a drug injection device. The self-sealing septum may be arranged such that an insertion of the head into the skirt cavity seals the open outlet end portion around the rear needle portion. Specifically, the self-sealing septum may be arranged at a transversal wall connecting the needle hub member and the skirt member.

The needle assembly may further comprise means for retaining the head in the skirt cavity. In an exemplary embodiment thereof, the head has a head configuration, the skirt member comprises a radially inwardly protruding structure, such as e.g. a lip member, whereby a skirt cavity configuration is formed in the skirt member between the self-sealing septum and the radially inwardly protruding structure, and the skirt cavity configuration corresponds to the head configuration. Thereby, when the head is inserted into the skirt cavity the inwardly protruding structure holds the head firmly in place, providing a tight fitting of the needle assembly on the drug cartridge and sealing the open outlet end portion around the rear needle portion, which itself provides permanent fluid connection to the interior of the drug cartridge.

This allows for a simple and compact needle system which can be swiftly and easily inserted into an injection device, e.g. by sliding or snapping action, in a ready to use state, requiring no additional handling steps. Furthermore, the self-sealing septum can be affixed to the drug cartridge without the need for an additional end cap, reducing the number of components constituting the needle system. The needle assembly may further comprise a removable or non-removable plug member as described above.

The skirt cavity has a size and a shape, and the head has a size and a shape, and in the present context, the phrase “the skirt cavity configuration corresponds to the head configuration” means that the size and shape of the skirt cavity equals the size and shape of the head. However, for embodiments where the skirt member comprises a pre-arranged self-sealing septum the phrase also implicitly covers the skirt cavity configuration being slightly smaller than the head configuration such that the head compresses the self-sealing septum during entry into the skirt cavity to thereby increase the sealing capability of the self-sealing septum along a distal rim of the head.

In a third aspect the invention provides a method of manufacturing a needle system comprising a needle assembly and a drug cartridge, the method comprising i) providing a needle assembly comprising a needle hub member in which an injection needle is fixedly mounted such that a front needle portion extends in a distal direction therefrom and a rear needle portion extends in a proximal direction therefrom, a skirt member extending proximally from the needle hub member and defining a skirt cavity, and a self-sealing septum, wherein the rear needle portion extends into the skirt cavity through the self-sealing septum and the skirt member comprises a radially inwardly protruding structure such that a skirt cavity configuration is formed in the skirt member between the self-sealing septum and the radially inwardly protruding structure, ii) providing a drug cartridge comprising a generally cylindrical body being sealed proximally by a slidable piston and having an open outlet end portion defined by a neck and a head, wherein the head has a head configuration which corresponds to the skirt cavity configuration, and iii) mounting the skirt member on the head.

For the avoidance of any doubt, in the present context the term “injection device” designates an apparatus suitable for injecting fluid media into the body of a subject, e.g. with the aid of an attachable needle device, and the term “drug” designates a medium which is used in the treatment, prevention or diagnosis of a condition, i.e. including a medium having a therapeutic or metabolic effect in the body. Further, the terms “distal” and “proximal” denote positions at, or directions along, a drug delivery device, a drug reservoir, or a needle unit, where “distal” refers to the drug outlet end and “proximal” refers to the end opposite the drug outlet end.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 is a longitudinal section view of a needle system according to an exemplary embodiment of the invention,

FIG. 2 is a longitudinal section view of a needle system according to another exemplary embodiment of the invention,

FIG. 3 is a v-section perspective view of a needle assembly for use in a needle system according to yet another exemplary embodiment of the invention,

FIGS. 4 and 5 are longitudinal section views of a needle system employing the needle assembly of FIG. 3,

FIG. 6 is a perspective view of the needle system inserted in a cartridge holder forming part of an injection device,

FIGS. 7-9 are perspective views of the needle assembly in different states during use, and

FIGS. 10 and 11 are longitudinal section views of a needle system according to a further exemplary embodiment of the invention.

In the figures like structures are mainly identified by like reference numerals.

### DESCRIPTION OF EXEMPLARY EMBODIMENTS

When/If relative expressions, such as “upper” and “lower”, “left” and “right”, “horizontal” and “vertical”, “clockwise” and “counter-clockwise”, etc., are used in the following, these refer to the appended figures and not necessarily to an actual situation of use. The shown figures are schematic representations for which reason the configuration of the different structures as well as their relative dimensions are intended to serve illustrative purposes only.

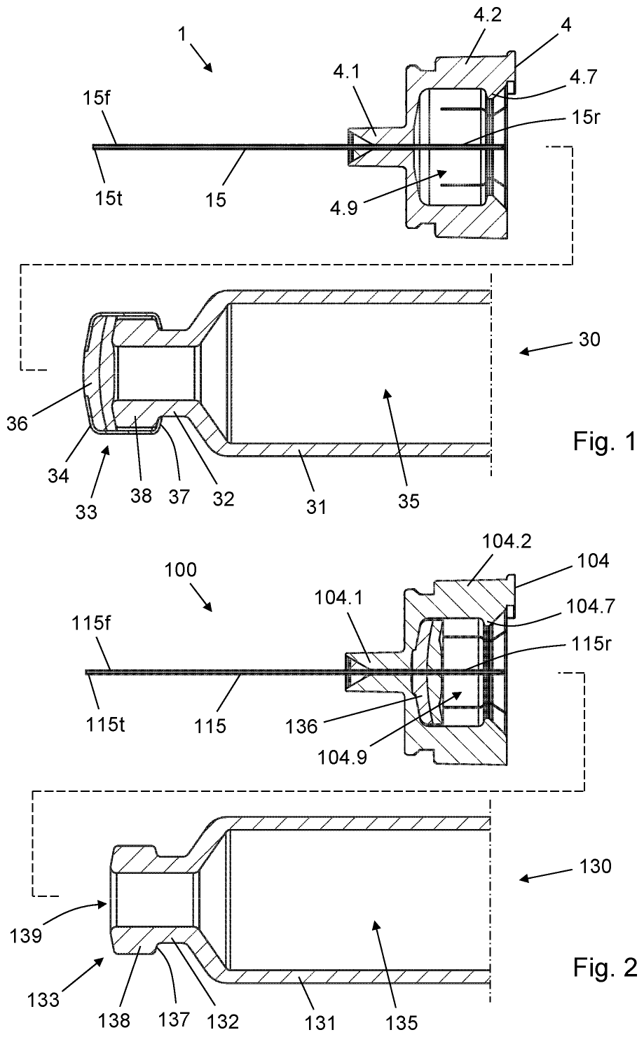

FIG. 1 is a longitudinal section view of a needle system according to a first exemplary embodiment of the invention, in the form of a needle assembly 1 and a drug cartridge 30 (of which only a distal portion is shown). The needle assembly 1 comprises a needle carrier 4 and an injection needle 15. The injection needle 15 is fixated in a needle hub 4.1, which forms a distal end portion of the needle carrier 4, such that a front needle portion 15f extends in a distal direction therefrom and a rear needle portion 15r extends in a proximal direction therefrom. The front needle portion 15f is provided with a sharpened front needle tip 15t for easy insertion through a skin barrier. A skirt 4.2 formed in one with the needle hub 4.1 has a circumferential interior lip 4.7 and is shaped provide a cavity 4.9 with a cavity configuration distally of the interior lip 4.7. The rear needle portion 15r extends into, and partly beyond, the cavity 4.9.

The drug cartridge 30 comprises a generally cylindrical cartridge body 31, with a neck 32 and a collar 38 at its distal most portion, defining an outlet end. The outlet end is sealed by a penetrable self-sealing septum 36 which is immobilised on the collar 38 by a ferrule 34. The ferrule 34 is bent around, and thereby fastened behind, a proximal rim 37 of the collar 38 such that the collar 38, the self-sealing septum 36 and the ferrule 34 together constitute a cartridge head 33 having a head configuration. The cartridge body 31 is proximally sealed by a slidable rubber piston (not shown), and the drug cartridge 30 thus comprises a confined chamber 35 which holds a liquid drug substance (not visible).

The cavity configuration corresponds to the head configuration, i.e. the cartridge head 33 has a size and a shape which exactly fits into the cavity 4.9, such that when the skirt 4.2 is mounted on the cartridge head 33 the interior lip 4.7 snaps in behind the portion of the ferrule 34 which is bent around the proximal rim 37. Thereby, a tight fitting of the needle assembly 1 on the drug cartridge 30 is obtained, which is both firm and stable, as no space for wriggling is present. Also, the rear needle portion 15r penetrates the self-sealing septum 36 and resides permanently in the chamber 35. Hence, a fluid connection between the chamber 35 and the injection needle 15 is pre-established in the needle system which is consequently ready for injection upon insertion into a dedicated injection device (not shown).

The needle system is simple and made of few components which renders it inexpensive to produce. Furthermore, the needle assembly comprises no movable parts and the needle system is thus functionally very reliable.

A removable plug (not shown) can optionally be included to seal off the front needle portion 15f. Such a plug could e.g. be made of an elastomeric material and structured to provide for easy removal by sliding motion along an exterior surface of the injection needle 15.

FIG. 2 is a longitudinal section view of a needle system according to a second exemplary embodiment of the invention, in the form of a needle assembly 100 and a drug cartridge 130 (of which only a distal portion is shown). The needle assembly 100 comprises a needle carrier 104 and an injection needle 115. The injection needle 115 is fixated in a needle hub 104.1, which forms a distal end portion of the needle carrier 104, such that a front needle portion 115f extends in a distal direction therefrom and a rear needle portion 115r extends in a proximal direction therefrom. The front needle portion 115f is provided with a sharpened front needle tip 115t for easy insertion through a skin barrier. A skirt 104.2 formed in one with the needle hub 104.1 has a circumferential interior lip 104.7 at a proximal end portion and a self-sealing septum 136 pre-arranged at a distal end portion. The skirt 104.2 thus defines a cavity 104.9, between the self-sealing septum 136 and the interior lip 104.7, which has a cavity configuration. The rear needle portion 115r extends through the self-sealing septum 136, into, and partly beyond, the cavity 104.9.

The drug cartridge 130 comprises a generally cylindrical cartridge body 131, with a neck 132 and a collar 138 at its distal most portion, defining an outlet end. The cartridge body 131 is proximally sealed by a slidable rubber piston (not shown), but in contrast to the first embodiment the outlet end is open and the drug cartridge 130 thus comprises an open chamber 135 which holds a liquid drug substance (not visible). In this case the collar 138 alone constitutes a cartridge head 133 having a head configuration.

The cavity configuration corresponds to the head configuration, i.e. the cartridge head 133 has a size and a shape which fits into the cavity 104.9, such that when the skirt 104.2 is mounted on the cartridge head 133 the interior lip 104.7 snaps in behind the proximal rim 137. Notably, in this case the fact that the cavity configuration corresponds to the head configuration actually comprises the cavity configuration being slightly smaller than the head configuration to account for any compressibility of the self-sealing septum 136. Such compressibility will enhance the sealing of the drug cartridge 130 when the needle assembly 100 is mounted thereon, and the cartridge head 133 may therefore deform the self-sealing septum slightly upon entry into the cavity 104.9.

A tight fitting of the needle assembly 100 on the drug cartridge 130 is thereby obtained, which is both firm and stable, as no space for wriggling is present. With this solution a ferrule for immobilising the self-sealing septum 136 can even be omitted, reducing the number of components of the needle system, because the skirt member 104.2 effectively takes over the function of the ferrule as the interior lip 104.7 snaps in behind the proximal rim 137 to maintain close contact between the cartridge head 133 and the self-sealing septum 136.

Also, the rear needle portion 115r resides permanently in the chamber 135. Hence, a fluid connection between the chamber 135 and the injection needle 115 is pre-established in the needle system which is consequently ready for injection upon insertion into a dedicated injection device (not shown). The needle system is simple and made of few components which renders it inexpensive to produce. Furthermore, the needle assembly comprises no movable parts and the needle system is thus functionally very reliable.

A removable plug (not shown) can optionally be included to seal off the front needle portion 115f. Such a plug could e.g. be made of an elastomeric material and structured to provide for easy removal by sliding motion along an exterior surface of the injection needle 115.

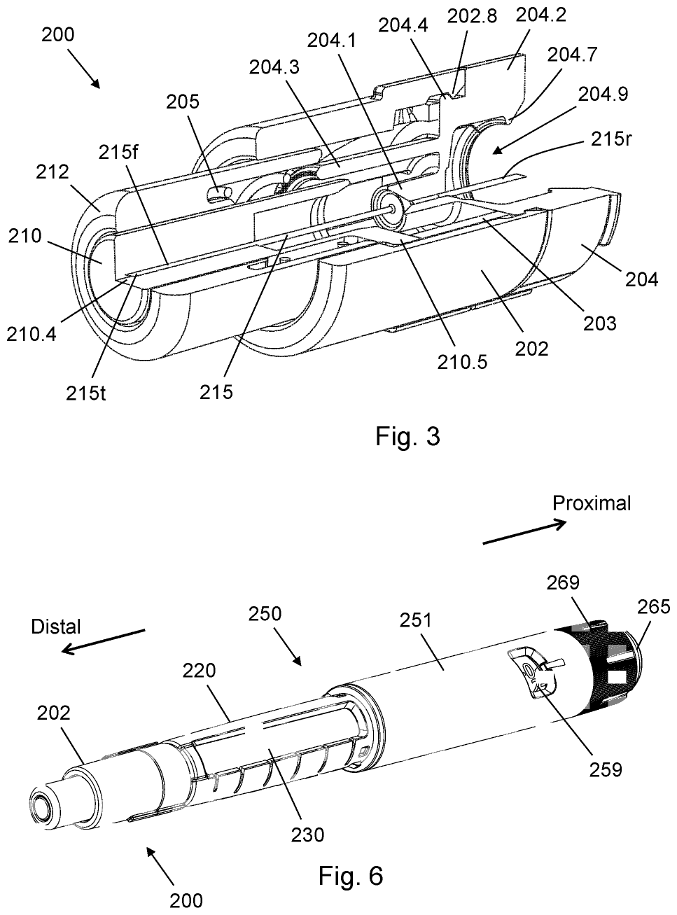

FIG. 3 is a perspective view of a needle assembly 200 for a needle system according to a third exemplary embodiment of the invention. The needle assembly 200 is shown in a pre-use state, sectioned by a 90° cut.

The needle assembly 200 comprises a needle carrier 204 and an injection needle 215. The injection needle 215 is fixated in a needle hub 204.1, which forms a distal portion of the needle carrier 204, such that a front needle portion 215f extends in a distal direction therefrom and a rear needle portion 215r extends in a proximal direction therefrom. The front needle portion 215f is provided with a sharpened front needle tip 215t for easy insertion through a skin barrier. A skirt 204.2 formed in one with the needle hub 204.1 has a circumferential interior lip 204.7 and is shaped provide a cavity 204.9 with a cavity configuration distally of the interior lip 204.7. The rear needle portion 215r extends into, and partly beyond, the cavity 204.9.

At the junction between the needle hub 204.1 and the skirt 204.2 the needle carrier 204 is provided with a circumferential flange 204.4 which serves as a means for attachment of an outer track sleeve 202. The outer track sleeve 202 is arranged concentrically about the needle hub 204.1 and has an interior rim 202.8 which is snapped behind the circumferential flange 204.4. An inner track sleeve 203 is arranged within the outer track sleeve 202 and the two are both axially and rotationally interlocked, functioning as a single unit.

The needle assembly 200 further comprises a plug 210 fitted snugly around the front needle portion 215f. The plug 210 is biostatic in that it is made of a thermoplastic elastomer containing immobilised Zinc (Zi⁺⁺) for neutralising micro-bacterial contaminants and it has a self-sealing front section 210.4 and a pair of opposite plug arms 210.5 (only one is visible).

Extending distally from the junction between the needle hub 204.1 and the skirt 204.2 a pair of plug guides 204.3 (only one is visible) arranged opposite to one another form axial slots (not visible) in which the respective plug arms 210.5 are slidably received. The plug 210 is thus rotationally fixed but axially displaceable with respect to the needle carrier 204.

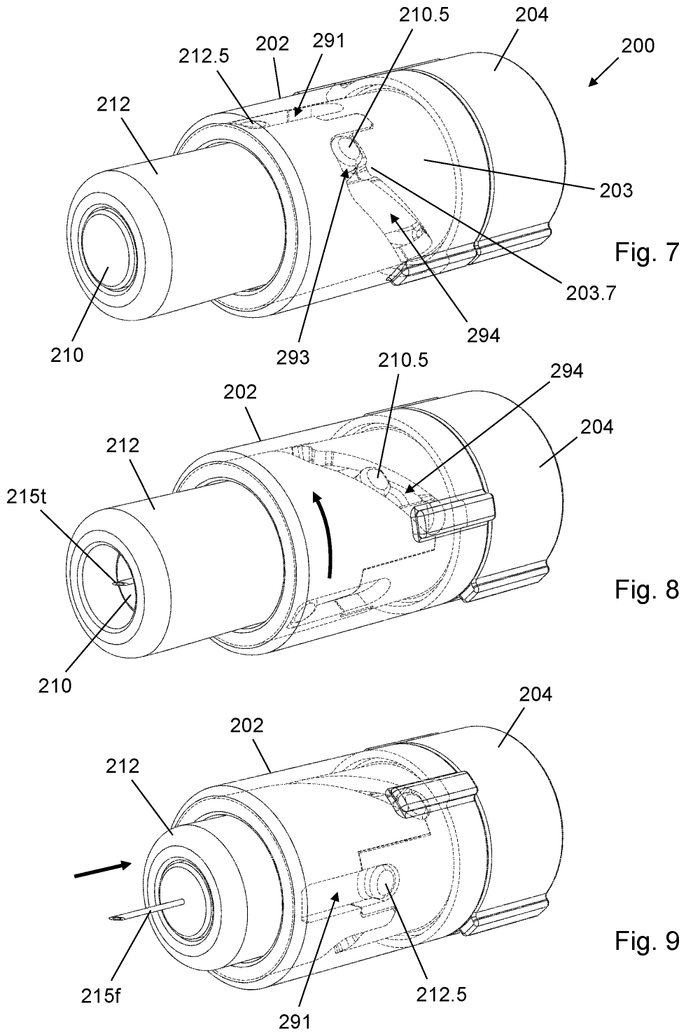

A needle shield 212 is arranged about the plug 210 and is axially displaceable relative to the needle carrier 204 between a needle covering position, in which a distal end portion of the needle shield 212 extends beyond and thereby covers the front needle tip 215t (as depicted in FIG. 3), and a needle exposing position in which the front needle tip 215t extends beyond the distal end portion of the needle shield 212. The needle shield 212, which is provided with a pair of diametrically opposite radial protrusions 212.5 (ref. FIGS. 7-9) is biased towards the needle covering position by a shield spring 205.

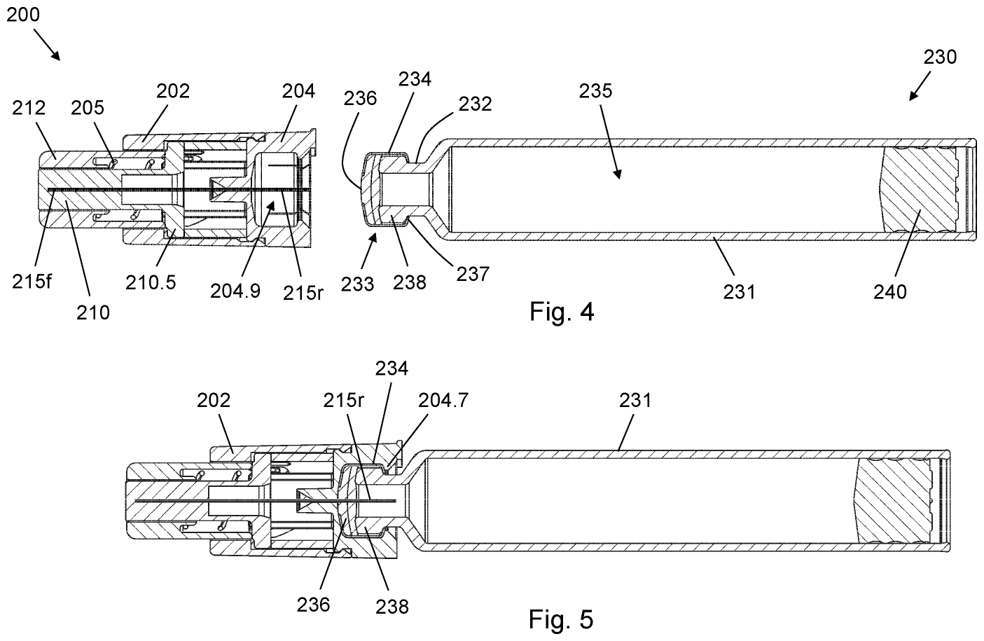

FIG. 4 is a longitudinal section view of the needle system, comprising the needle assembly 200 and a drug cartridge 230, in a pre-assembled state. The drug cartridge 230 is identical to the drug cartridge 30 of the first embodiment of the present invention, i.e. it comprises a generally cylindrical cartridge body 231, with a neck 232 and a collar 238 at its distal most portion, defining an outlet end. The outlet end is sealed by a penetrable self-sealing septum 236 which is immobilised on the collar 238 by a ferrule 234. The ferrule 234 is bent around, and thereby fastened behind, a proximal rim 237 of the collar 238 such that the collar 238, the self-sealing septum 236 and the ferrule 234 together constitute a cartridge head 233 having a head configuration. The cartridge body 231 is proximally sealed by a slidable rubber piston 240, and the drug cartridge 230 thus comprises a confined chamber 235 which holds a liquid drug substance (not visible).

The cavity configuration corresponds to the head configuration, i.e. the cartridge head 233 has a size and a shape which exactly fits into the cavity 204.9, such that when the skirt 204.2 is mounted on the cartridge head 233 the interior lip 204.7 snaps in behind the portion of the ferrule 234 which is bent around the proximal rim 237. Thereby, a tight fitting of the needle assembly 200 on the drug cartridge 230 is obtained, which is both firm and stable, as no space for wriggling is present. Also, the rear needle portion 215r penetrates the self-sealing septum 236 and resides permanently in the chamber 235. This is illustrated in FIG. 5 which shows the needle system in an assembled state.

Hence, a fluid connection between the chamber 235 and the injection needle 215 is pre-established in the needle system which is consequently ready for injection upon insertion into a dedicated injection device 250, as shown in FIG. 6. The injection device 250 comprises a housing 251 which accommodates a dose setting mechanism operable by rotation of a dose dial 269 and a dose expelling mechanism operable by depression of an injection button 265. A scale drum 259 for indicating the size of a current set dose is viewable through a window in the housing 251.

The injection system can be transversally snap fitted into a cartridge holder 220 (from the side opposite to the one visible in the figure) which is permanently attached to the housing 251 in axial extension thereof, and the injection device 250 can thereby by readied for use quickly and easily.

FIGS. 7-9 show how the needle assembly 200 is operated during use of the injection device 250. For the sake of clarity, the injection device 250 has been omitted from the figures but it is implicit that when the needle assembly is operated as described below the skirt 204.2 is mounted tightly on the cartridge head 233 and the rear needle portion 215r resides in the chamber 235. Also, the outer track sleeve 202 is depicted as a see-through component to allow presentation of interior track configurations (indicated with dotted lines) formed by interior geometries of the outer track sleeve 202 and an exterior shape of the inner track sleeve 203.

In FIG. 7 the needle assembly 200 is in a pre-use, or between-use, state where the needle shield 212 is maintained in the needle covering position by the shield spring 205 and the plug 210 covers the front needle tip 215t. The injection needle 215 is thereby sealed off and no liquid can escape the chamber 235. As mentioned, the outer track sleeve 202 and the inner track sleeve 203 together form interior track configurations. These serve to guide respective movements of the plug 210 and the needle sleeve 212 relative to the needle carrier 204.

Specifically, the interior track configurations comprise a pair of diametrically opposite needle shield tracks 291 for sliding accommodation of the respective radial protrusions 212.5, and a pair of diametrically opposite plug tracks (only one is visible) for sliding accommodation of the respective plug arms 210.5. The needle shield tracks 291 extend axially along a longitudinal reference axis of the injection needle 215, and each of the plug tracks comprise a circumferential track section 293 and a helical track section 294. The circumferential track section 293 and the helical track section 294 are connected at a track narrowing formed by a bulb 203.7. In the shown state of the needle assembly 200 the plug arms 210.5 rest in the respective circumferential track sections 293 and the radial protrusions 212.5 are positioned at respective distal ends of the needle shield tracks 291. Both the plug 210 and the needle shield 212 are thereby axially locked with respect to the needle carrier 204, safely covering the front needle tip 215t and preventing accidental needle stick injuries.

In the following, with reference to FIGS. 8 and 9, only the movements of one plug arm 210.5 and one radial protrusion 212.5 are described, it being understood that the other plug arm 210.5 and the other radial protrusion 212.5 respectively undergo similar movements.

Firstly, the user grabs the needle carrier 204 with one hand and rotates the outer track sleeve 202 counter-clockwise (seen from a distal perspective) relative to the needle carrier 204 with the other hand, i.e. in the direction of the arrow shown in FIG. 8. By the user initially applying a relatively high torque the plug arm 210.5 elastically deforms as it passes the bulb 203.7 and enters the helical track section 294. The relatively high torque needed to move the plug arm 210.5 out of the circumferential track section 293 is advantageous from a safety perspective as it reduces the risk of premature release of the plug 210 and the needle shield 212, which could otherwise occur accidentally if e.g. the injection device 250 lying in a purse or bag were subjected to impact from neighbouring objects.

Continuing the rotation of the outer track sleeve 202 relative to the needle carrier 204, now applying a lower torque, the user forces the plug arm 210.5 down the helical track section 294, and thereby proximally in the axial slots formed by the plug guides 204.3. The plug 210 is thus pulled proximally, eventually exposing the front needle portion 215f within the needle shield 212.

When the plug arm 210.5 reaches a proximal end of the helical track section 294 the user places the needle shield 212 on a skin surface and presses the needle carrier 204 towards the body. A proximally directed force from the skin is thereby applied to the needle shield 212 which consequently slides from the needle covering shield position to the needle exposing shield position as the radial protrusion 212.5 travels the axial needle shield track 291 against the force from the shield spring 205. This is shown in FIG. 9. The front needle portion 15f thus enters the skin and the user can perform an injection of a set dose of drug by operation of the injection button 265.

Upon completion of the injection the user pulls the front needle portion 215f out of the skin, whereby the shield spring 205 returns the needle shield 212 to the needle covering shield position, as the radial protrusion 212.5 return in the axial needle shield track 291. Subsequently, the user rotates the outer track sleeve 202 clockwise relative to the needle carrier 204 to return the plug arm 210.5 along the helical track section 294 to the circumferential track section 293. Again, a relatively high torque is needed for the plug arm 210.5 to pass the bulb 203.7, but once the plug arm 210.5 is re-seated in the circumferential track section 293 the needle assembly 200 is safely locked, preventing any accidental needle stick injuries. This corresponds to the state shown in FIG. 7.

Since the plug 210 comprises a micro-bacterial growth inhibitor the needle system is suitable for multiple use, as the front needle portion 215f housed in the plug 210 is cleaned between injection actions. In particular, a needle system is thereby provided where the use time of the needle assembly 200 equals that of the drug cartridge 230, such that the user only needs to change the needle assembly 200 when the drug cartridge 230 is exhausted. This markedly reduces the number of handling steps which the user must go through over time to prepare for injections with the injection device 250.

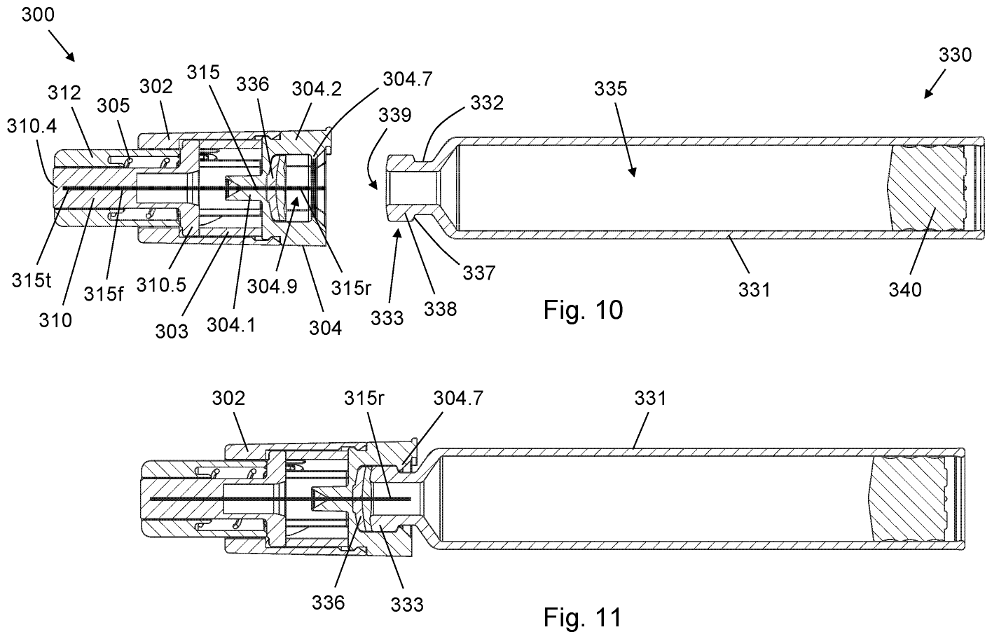

FIGS. 10 and 11 are longitudinal section views of a needle system according to a fourth exemplary embodiment of the invention, comprising a needle assembly 300 and a drug cartridge 330, where FIG. 10 shows the needle system in a pre-assembled state and FIG. 11 shows the needle system in an assembled state.

The needle assembly 300 is substantially identical to the needle assembly 200 described above in connection with the third embodiment of the invention, i.e. it comprises a needle carrier 304 with a needle hub 304.1 and a skirt 304.2, the needle hub 304.1 carrying an injection needle 315 with a rear needle portion 315r, and the skirt 304.2 being provided with an interior lip 304.7, a front needle portion 315f, and a front needle tip 315t, an outer track sleeve 302, an inner track sleeve 303, a plug 310 with a self-sealing front section 310.4 and a pair of plug arms 310.5, and a needle shield 312 which is biased distally by a shield spring 305.

The only structural difference between the needle assembly 300 and the needle assembly 200 is the provision of a self-sealing septum 336, pre-arranged within the skirt 304.2 just proximally of the needle hub 304.1. The self-sealing septum 336 is permanently transpierced by the rear needle portion 315r but forms a sealing lip around a portion thereof. The skirt 304.2 thus defines a cavity 304.9, between the self-sealing septum 336 and the interior lip 304.7, which has a cavity configuration.

The drug cartridge 330 is identical to the drug cartridge 130 of the second embodiment of the present invention, i.e. it comprises a generally cylindrical cartridge body 331, with a neck 332 and a collar 338 at its distal most portion, defining an outlet end. The cartridge body 331 is proximally sealed by a slidable rubber piston 340, but in contrast to the former embodiment the outlet end is open and the drug cartridge 330 thus comprises an open chamber 335 which holds a liquid drug substance (not visible). In this case the collar 338 alone constitutes a cartridge head 333 having a head configuration.

The cavity configuration corresponds to the head configuration, i.e. the cartridge head 333 has a size and a shape which exactly fits into the cavity 304.9, such that when the skirt 304.2 is mounted on the cartridge head 333, as shown on FIG. 11, the interior lip 304.7 snaps in behind the proximal rim 337. Notably, in this case the fact that the cavity configuration corresponds to the head configuration actually comprises the cavity configuration being slightly smaller than the head configuration to account for any compressibility of the self-sealing septum 336. Such compressibility will enhance the sealing of the drug cartridge 330 when the needle assembly 300 is mounted thereon, and the cartridge head 333 may therefore deform the self-sealing septum slightly upon entry into the cavity 304.9.

A tight fitting of the needle assembly 300 on the drug cartridge 330 is thereby obtained, which is both firm and stable, as no space for wriggling is present. With this solution a ferrule for immobilising the self-sealing septum 336 can be omitted, reducing the number of components of the needle system compared to that of the third embodiment of the invention, because the skirt member 304.2 effectively takes over the function of the ferrule as the interior lip 304.7 snaps in behind the proximal rim 337 to maintain close contact between the cartridge head 333 and the self-sealing septum 336.

Also, the rear needle portion 315r resides permanently in the chamber 335. Hence, a fluid connection between the chamber 335 and the injection needle 315 is pre-established in the needle system which is consequently ready for injection upon insertion into a dedicated injection device, e.g. like the one shown in FIG. 6. In the assembled state of the needle system the needle assembly 300 is operated identically to the needle assembly 200 described above.

1. A needle system comprising a needle assembly and a drug cartridge, the needle assembly comprising:
a needle hub member in which an injection needle is fixedly mounted such that a front needle portion extends in a distal direction therefrom and a rear needle portion extends in a proximal direction therefrom, and
a skirt member extending proximally from the needle hub member and defining a skirt cavity accommodating the rear needle portion, the skirt cavity having a skirt cavity configuration, and
the drug cartridge comprising a generally cylindrical body being sealed proximally by a slidable piston and having an outlet end portion defined by a neck and a head, the head having a head configuration,
wherein the skirt cavity configuration corresponds to the head configuration, providing for a tight fitting of the skirt member on the head which renders the rear needle portion a permanent resident of the outlet end portion.

2. The needle system according to claim 1, wherein the skirt member and the needle hub member are formed in one piece.

3. The needle system according to claim 1, wherein the head comprises a self-sealing septum sealing the outlet end portion, and an end cap for immobilising the self-sealing septum.

4. The needle system according to claim 1, wherein the skirt member further comprises a pre-arranged self-sealing septum, and wherein the rear needle portion transpierces a central portion of the self-sealing septum.

5. The needle system according to claim 4, wherein the penetrable self-sealing septum is adapted to sealingly close the outlet end portion, when the skirt member is fitted on the head.

6. The needle system according to claim 1, wherein the skirt member is configured to snap to the drug cartridge behind the head.

7. The needle system according to claim 6, wherein the head comprises a trailing rim, and wherein the skirt member comprises a catch portion adapted to engage with the trailing rim.

8. The needle system according to claim 1, further comprising a removable plug member fitted tightly around a distal end of the front needle portion.

9. The needle system according to claim 1, wherein the injection needle extends along a reference axis, and wherein the needle assembly further comprises a plug member fitted tightly around a portion of the front needle portion, the plug member comprising a penetrable self-sealing front section and being axially displaceable with respect to the needle hub between a forward plug position in which a distal end of the front needle portion is housed within the plug member, proximally of the penetrable self-sealing front section, and a rearward plug position in which the distal end is exposed.

10. The needle system according to claim 9, wherein the plug member comprises a micro-bacterial growth inhibitor.
